(19) United States
(12) Patent Application Publication
(10) Pub. No.: US 2007/0219469 A1
(43) Pub. Date: Sep. 20, 2007

(54) Palpitation monitor

(76) Inventor: Terence Vardy, Tweed Heads (AU)

Correspondence Address: Martin P. Hoffman;HOFFMAN, WASSON & GITLER, P.C., Suite 522, 2461 South Clark Street, Arlington, VA, 22202, US

(21) Appl. No.: 11/723,037

(22) Filed: Mar. 16, 2007

(30) Foreign Application Priority Data
Mar. 16, 2006 (AU) 2006901381

(51) Int. Cl. A61B 5/103

(52) U.S. Cl. 600/587

(57) ABSTRACT

A method for the therapeutic treatment of a patient which enables consistent diagnosis and manual manipulative treatment of a patient by a therapist. The therapist wears pressure sensors on pressure zones, such as fingers and thumbs, and the sensors are connected to a monitor. The monitor provides feedback to the therapist with respect to the pressure applied to the patient.

### FIELD OF THE INVENTION

The present invention pertains to a method for palpatory diagnosis and the therapeutic treatment of a patient, the method employing pressure sensors worn by the therapist on pressure zones defined on the hands of the therapist.

### BACKGROUND OF THE INVENTION

The present invention relates to the use of palpatory-sensory-feedback as a method for palpatory diagnosis and the therapeutic treatment of a patient, documentation of manual medicine treatment methods used, examination of medical students as to their proficiency in manual medicine, teaching and examination of medical students in surgical techniques, the use of palpatory feedback in hazardous activities such as bomb disposal activities, the application of such palpatory feedback devices for robotics to creates fine force applications, and the use of feedback sensors for prostheses' force determination.

Many manual medicine treatments involve the palpatory diagnosis and manual manipulation of a patient by a therapist. Therapists are usually trained in such diagnosis and manual manipulations and learn, over a period of time, the pressures that should be applied to a patient.

There is often considerable variation in palpatory diagnosis and the manual manipulation of a patient between different therapists seeking to apply the same, or similar diagnosis and treatment. In addition, there can also be considerable variation in the pressure applied to a patient by a single therapist during different treatment sessions. It will be appreciated that the manual manipulation of a patient is generally subject to considerable variation.

### BRIEF SUMMARY OF THE INVENTION

We have now found a method for the therapeutic treatment of a patient which enables a more consistent diagnosis and manual manipulative treatment of a patient between therapists and between sessions. Accordingly the present invention provides a method for diagnosis and therapeutic treatment of a patient comprising manual manipulation of the patient by a therapist wherein said therapist wears pressure sensors on pressure zones, said pressure sensors being connected to a monitor.

It will be appreciated that the therapist's pressure zones will depend upon the manual manipulation treatment to which the patient is being subjected. In many treatments and diagnosis, the pressure zones of the therapist will be the therapist's fingers and thumbs. In such diagnosis and treatments, the pressure sensors will be applied to the fingers and thumbs of the therapist. In other treatments, the therapist may apply pressure to the patient using the eminence of the hand, the forearm, the elbow or other zones of the therapist's body. Pressure sensors may be worn by the therapist over those zones in order to measure the pressure applied by the therapist to the patient.

The pressure sensors may be of any convenient configuration that can be connected to a monitor. For example, the pressure sensors may be selected from the group consisting of pressure transducers, load cells, pyroelectric sensors and the like.

The pressure sensors may be worn by the therapist or attached to a prosthesis or robotic arm by attaching the pressure sensors to gloves, finger pouches, patches such as may be readily attached to the therapist's clothing (e.g., by hook and loop fastener). The pressure sensors may be attached to an electrical connection on the therapist such as clipped to the therapist's belt or on a suitable vest configured to house the electrical connections and provide a display at such point as at the wrist or on the forearm.

In a first embodiment of the present invention, the method may be used to provide feedback to the therapist in order to allow the therapist to regulate the forces applied to the patient during diagnosis and manual manipulation. In this first embodiment, the present invention provides a method for diagnosis and therapeutic treatment of a patient comprising manual manipulation of the patient by a therapist wherein said therapist wears pressure sensors on pressure zones, said pressure sensors being connected to a monitor wherein said monitor provides feedback to the therapist with respect to the pressure applied to the patient during manual manipulation and diagnosis.

In this first embodiment of the present invention, the feedback may be in the form of an audio signal which instructs the therapist to provide more or less pressure to the patient. It is also envisaged that a visual feedback may be provided. One of the advantages of providing a visual feedback may be that it is possible to display feedback for individual pressure zones so that not only is the total pressure being optimized by also the pressure applied from individual zones. In a further embodiment, it is also envisaged that feedback may be directly provided to the individual pressure zones such as by electrical stimulation or other stimulation that may be sensed by touch. Again, by providing feedback specific to individual pressure zones, the balance of the force applied during palpatory diagnosis and manual manipulation may be controlled. Also that the required force to be supplied by a robotic arm or prosthesis may be governed by the sensor feedback acting with a software system capable of providing fine motor control skills.

We have found that the method of the present invention is also useful in monitoring and programming the diagnosis and therapeutic treatments of a patient. For example, by comparing the data from various treatments and diagnosis, it may be possible to design subsequent treatments to optimize the therapeutic effect. In this second embodiment of the present invention, there is provided a method for therapeutic treatment of a patient comprising manual manipulation of the patient by a therapist wherein said therapist wears pressure sensors on pressure zones, said pressure sensors being connected to a monitor, wherein said monitor includes a recorder for recording and comparing manipulations from a series of therapeutic treatments.

Advantageously, the data recorded in the second embodiment of the present invention may be used to review patient therapies or to program continued therapies in order to optimize the healing effect.

It will be appreciated that a variety of different electronic recording media and analytical means may be used to record and compare data from a series of therapeutic treatments.

We have also found that the method of the present invention is particularly suited for training therapists in a variety of manual manipulation treatments and diagnosis. According to a third embodiment of the present invention there is provided a method for therapeutic treatment of a patient comprising manual manipulation of the patient by a therapist wherein said therapist wears pressure sensors on pressure zones, said pressure sensors being connected to a monitor wherein said monitor provides feedback to the therapist whereby said therapist is trained in techniques for the manual manipulation of the patient and diagnosis.

The present invention will now be described with reference to the accompanying drawings which have been included to illustrate the invention and are not intended to limit the invention in any way.

### BRIEF DESCRIPTION OF THE DRAWINGS

FIG. 1 shows a pair of therapists wearing sensors in accordance with the present invention.

FIG. 2 shows a therapist manually manipulating a patient.

FIG. 3 shows a vest carrying the wiring looms for the pressure sensors.

FIG. 4 shows the vest of FIG. 1 connected to a monitor.

### DETAILED DESCRIPTION OF THE INVENTION

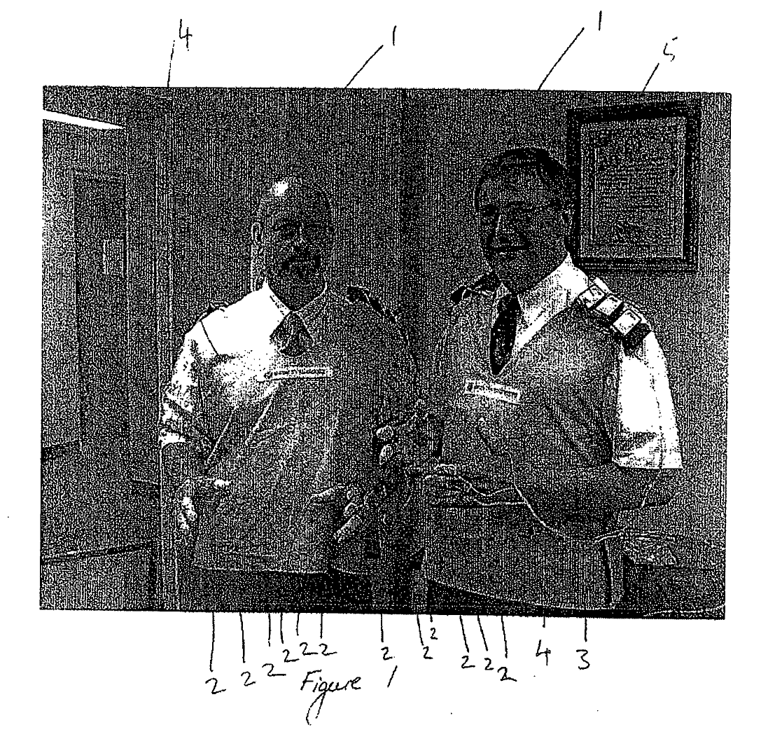

The therapists 1 shown in FIG. 1 are wearing sensors 2 on their respective thumbs, index and middle fingers. The sensors 2 are retained on the therapist's fingers by latex sleeves. The pressure sensors 2 are connected to a vest 4 which supports electrical connections 5.

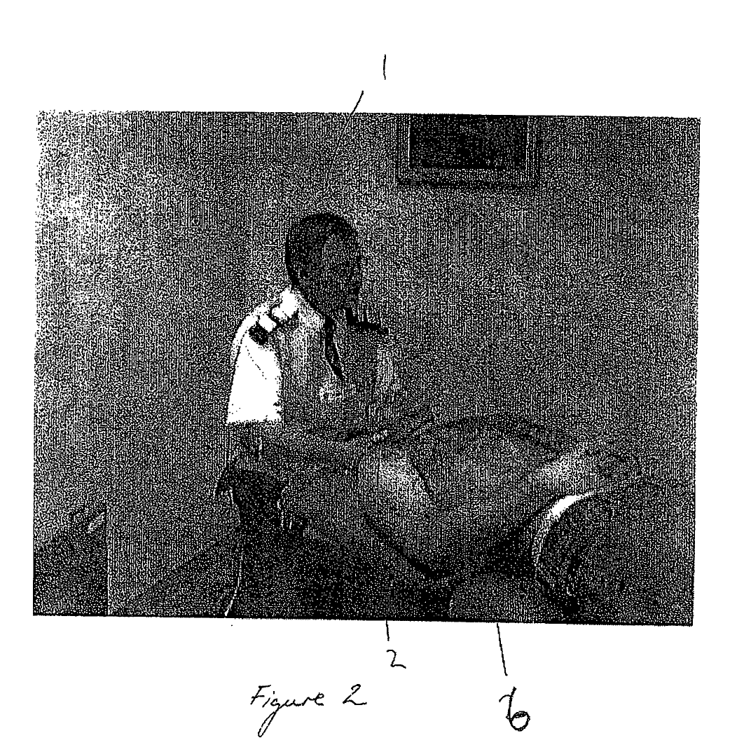

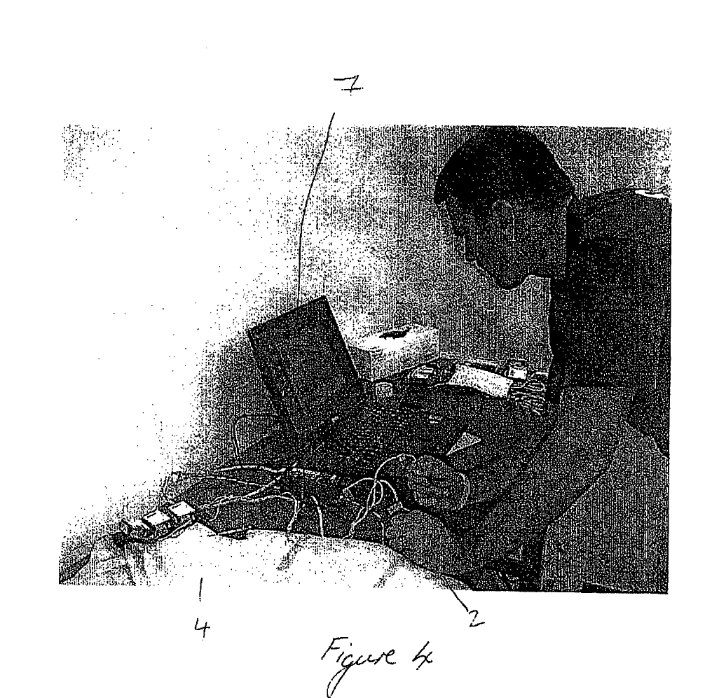

FIG. 2 shows a therapist 1 manipulating a patient 6. The therapist 1 applies force to the patient 2 via his hands which support the sensors 2. The applied force is measured on the monitor 7 as shown in FIG. 4. The measurement of the force applied to the patient 6 by the therapist 1 can be recorded over time and the data presented graphically for analysis.

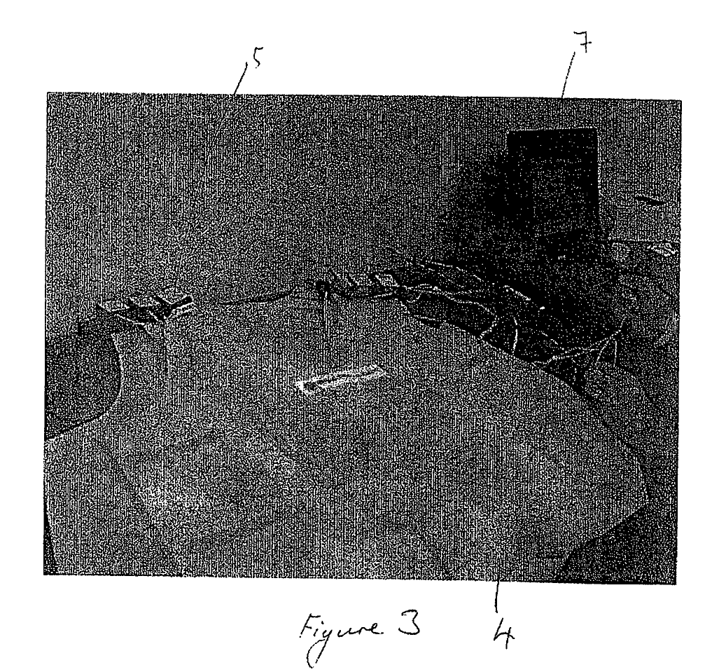

FIG. 3 shows a vest 4 which supports the electrical connections 5 between the sensors (not shown) and the monitor 7.

FIG. 4 shows the pressure sensors 2 being calibrated on the monitor 7 prior to their use in the manual manipulation of the patient.

A person skilled in the art will appreciate that the invention described above may be subject to improvements and modifications that will be apparent without departing from the spirit and scope of the invention described herein.

1. A method for diagnosis and therapeutic treatment of a patient comprising manual manipulation of the patient by a therapist wherein said therapist wears pressure sensors on pressure zones, said pressure sensors being connected to a monitor.

2. A method for diagnosis and therapeutic treatment of a patient according to claim 1 wherein the pressure sensors are selected from the group consisting of pressure transducers, load cells, and pyroelectric sensors.

3. A method for diagnosis and therapeutic treatment of a patient according to claim 1 wherein the pressure sensors are worn by the therapist or attached to a prosthesis or robotic arm by attaching the pressure sensors to gloves, finger pouches, or patches attached to the therapist's clothing.

4. A method for diagnosis and therapeutic treatment of a patient according to claim 1 wherein the pressure sensors are attached to an electrical connection on the therapist such as clipped to the therapist's belt or on a suitable vest configured to house the electrical connections and provide a display at such point as at the wrist or on the forearm.

5. A method for diagnosis and therapeutic treatment of a patient according to claim 1 wherein the method provides feedback to the therapist in order to allow the therapist to regulate the forces applied to the patient during diagnosis and manual manipulation.

6. A method for diagnosis and therapeutic treatment of a patient comprising manual manipulation of the patient by a therapist wherein said therapist wears pressure sensors on pressure zones, said pressure sensors being connected to a monitor wherein said monitor provides feedback to the therapist with respect to the pressure applied to the patient during manual manipulation and diagnosis.

7. A method for diagnosis and therapeutic treatment of a patient according to claim 6 wherein the feedback is in the form of an audio signal which instructs the therapist to provide more or less pressure to the patient.

8. A method for diagnosis and therapeutic treatment of a patient according to claim 6 wherein the feedback is in visual form.

9. A method for diagnosis and therapeutic treatment of a patient according to claim 6 wherein the feedback is directly provided to the individual pressure zones such as by electrical stimulation that may be sensed by touch.

10. A method for diagnosis and therapeutic treatment of a patient according to claim 6 wherein the required force to be supplied by a robotic arm or prosthesis is governed by the sensor feedback acting with a software system capable of providing fine motor control skills.

11. A method for therapeutic treatment of a patient comprising manual manipulation of the patient by a therapist wherein said therapist wears pressure sensors on pressure zones, said pressure sensors being connected to a monitor, wherein said monitor includes a recorded for recording and comparing manipulations from a series of therapeutic treatments.

12. A method for diagnosis and therapeutic treatment of a patient according to claim 11 wherein the method is used to review patient therapies or to program continued therapies in order to optimize the healing effect.

13. A method for therapeutic treatment of a patient comprising manual manipulation of the patient by a therapist wherein said therapist wears pressure sensors on pressure zones, said pressure sensors being connected to a monitor wherein said monitor provides feedback to the therapist whereby said therapist is trained in techniques for the manual manipulation of the patient and diagnosis.
